(19) United States
(12) Patent Application Publication
(10) Pub. No.: US 2023/0263807 A1
(43) Pub. Date: Aug. 24, 2023

(54) TREPROSTINIL ILOPROST COMBINATION THERAPY

(71) Applicant: United Therapeutics Corporation, Silver Spring, MD (US)

(72) Inventors: Patrick Poisson, Chapel Hill, NC (US); Ravi Patel, Cary, NC (US); Robert Stewart Gurley, Lake Oswego, OR (US); Elizabeth Anne French, Apex, NC (US)

(73) Assignee: United Therapeutics Corporation, Silver Spring, MD (US)

(21) Appl. No.: 18/106,782

(22) Filed: Feb. 7, 2023

Related U.S. Application Data
(60) Provisional application No. 63/307,919, filed on Feb. 8, 2022.

(51) Int. Cl. A61K 31/5578; A61K 31/5575; A61K 9/00; A61K 47/02; A61K 47/12

(52) U.S. Cl. CPC A61K 31/5578; A61K 31/5575; A61K 9/0078; A61K 47/02; A61K 47/12

(57) ABSTRACT

The disclosure relates to treatment of a disease, such as pulmonary hypertension, by inhalation with a combination of iloprost and treprostinil.

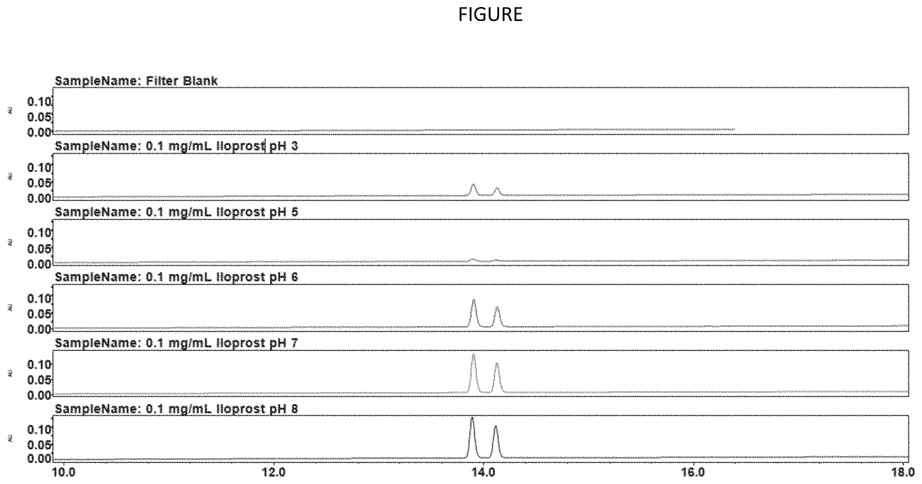

### RELATED APPLICATIONS

The present application claims priority to U.S. Provisional Patent Application No. 63/307,919 filed Feb. 8, 2022, which is incorporated herein by reference in its entirety.

### FIELD

The present application generally relates to compositions comprising prostacyclins and methods of treating a disease using prostacyclins, including compositions comprising treprostinil and iloprost and methods of treating a disease with a combination comprising treprostinil and iloprost.

### SUMMARY

One embodiment is a method of treating pulmonary hypertension comprising administering by inhalation to a subject in need thereof iloprost or a pharmaceutically acceptable salt thereof and treprostinil or a pharmaceutically acceptable salt thereof.

Yet another embodiment is a method of treating pulmonary hypertension comprising administering by inhalation to a subject in need thereof in a single event dose a composition comprising (i) iloprost or a pharmaceutically acceptable salt thereof and (ii) treprostinil or a pharmaceutically acceptable salt thereof, wherein the dosage of the iloprost or a pharmaceutically acceptable salt thereof delivered to the subject is at least 2.5 μg, and wherein the dosage of treprostinil or a pharmaceutically acceptable salt thereof delivered to the subject is at least 5 μg.

### FIGURES

The FIGURE shows chromatograms (arbitrary units versus minutes) of Iloprost solubility solutions at various pH levels.

### DETAILED DESCRIPTION

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either.” Thus, unless context indicates otherwise, the word “or” means any one member of a particular list and also includes any combination of members of that list. “Consisting of” shall mean excluding more than trace elements of other ingredients and substantial method steps. Embodiments defined by each of these transition terms are within the scope of this technology. When an embodiment is defined by one of these terms (e.g., “comprising”) it should be understood that this disclosure also includes alternative embodiments, such as “consisting essentially of” and “consisting of” for said embodiment.

All numerical designations, e.g., amount, time, and concentration, including ranges, are approximations which are varied (+) or (−) by increments of 0.05%, 1%, 2%, 5%, 10% or 20%. It is to be understood, although not always explicitly stated that all numerical designations are preceded by the term “about.”

“Subject” refers to an animal, such as a mammal (including a human), that has been or will be the object of treatment, observation or experiment. “Subject” and “patient” may be used interchangeably, unless otherwise indicated. The methods described herein may be useful in human therapy and/or veterinary applications. In some embodiments, the subject is a mammal. In some embodiments, the subject is a human.

Where a range of values is provided, it is understood that each intervening value, to the tenth of the unit of the lower limit unless the context clearly dictates otherwise, between the upper and lower limit of that range and any other stated or intervening value in that stated range, is encompassed within the present technology. The upper and lower limits of these smaller ranges may independently be included in the smaller ranges and are also encompassed within the present technology, subject to any specifically excluded limit in the stated range. Where the stated range includes one or both of the limits, ranges excluding either or both of those included limits are also included in the present technology.

“Pulmonary hypertension” (“PH”) refers to all forms of pulmonary hypertension including all pulmonary hypertension encompassed by WHO Groups 1-5 unless otherwise indicated or apparent to one of ordinary skill in the art. Pulmonary hypertension (PH) is a condition characterized by increased blood pressure in the arteries of the lungs. Symptoms of PH may include one or more of shortness of breath, fainting, tiredness, chest pain, swelling of the legs, and a fast heartbeat. “Pulmonary hypertension” includes pulmonary arterial hypertension (“PAH”) (Group 1) in all its forms, including idiopathic and heritable PAH. Pulmonary hypertension includes patients with NYHA Functional Class III symptoms. “Pulmonary hypertension” includes pulmonary hypertension due to left heart disease (Group 2). “Pulmonary hypertension” includes pulmonary hypertension due to lung disease (Group 3). “Pulmonary hypertension” includes thromboembolic pulmonary hypertension (Group 4). “Pulmonary hypertension” includes pulmonary hypertension secondary to other conditions, such as sarcoidosis, sickle cell anemia, chronic hemolytic anemia, splenectomy, and certain metabolic disorders. (Group 5). Generally, the methods of treatment described herein are most applicable to PAH (Group 1) and Group 3 pulmonary hypertension, including PH-ILD.

An embodiment is a method of treating pulmonary hypertension by administering by inhalation to a subject, preferably a human being suffering from pulmonary hypertension, a combination of iloprost and treprostinil. In place of or in addition to the treprostinil or iloprost, pharmaceutically acceptable salts or esters or prodrugs can be administered. For example, iloprost and the sodium salt of treprostinil can be administered to treat pulmonary hypertension. Unless otherwise indicated, references herein to treprostinil and iloprost include pharmaceutically acceptable salts, esters, and prodrugs of these compounds.

Iloprost (tradenames: Ventavis®, Ilomedine) is used to treat pulmonary arterial hypertension (PAH), scleroderma, Raynaud's phenomenon and other diseases in which the blood vessels are constricted and blood cannot flow to the tissues. Iloprost was developed by the pharmaceutical company Schering AG and is marketed by Bayer Schering Pharma AG in Europe and Actelion Pharmaceuticals in the USA. Iloprost is a synthetic analogue of prostacyclin PGI₂ having the following structure:

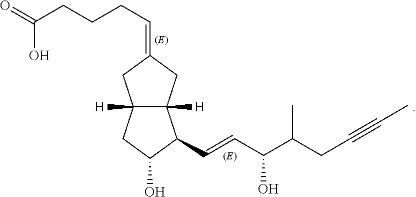 [SMILES: CC#CCC(C)[C@H](O)/C=C/[C@@H]1[C@H]2C/C(=C/CCCC(=O)O)C[C@H]2C[C@H]1O]

In the U.S., iloprost (Ventavis®) solution is approved for inhalation using the I-Neb AAD or Prodose AAD delivery systems. In Europe, iloprost as Ventavis has been approved for use with two compressed air nebulizers with AAD delivery systems (Halolite and Prodose) as well as with two ultrasonic nebulizers, Ventaneb and I-Neb.

Treprostinil is also used for the treatment of pulmonary arterial hypertension. Treprostinil is a synthetic analog of prostacyclin (PGI₂) having the following structure:

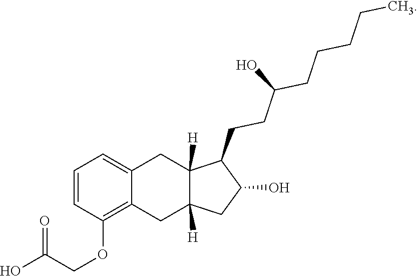 [SMILES: CCCCC[C@H](O)CC[C@@H]1[C@H]2Cc3cccc(OCC(=O)O)c3C[C@H]2C[C@H]1O]

Treprostinil, the active ingredient in Remodulin® (treprostinil) Injection, Tyvaso® (treprostinil) Inhalation Solution, and Orenitram® (treprostinil) Extended-Release Tablets, was described in U.S. Pat. No. 4,306,075. Methods of making treprostinil and other prostacyclin derivatives are described, for example, in Moriarty, et al., J. Org. Chem. 2004, 69, 1890-1902, Drug of the Future, 2001, 26(4), 364-374, U.S. Pat. Nos. 6,441,245, 6,528,688, 6,700,025, 6,809,223, 6,756,117, 8,461,393, 8,481,782; 8,242,305, 8,497,393, 8,940,930, 9,029,607, 9,156,786 and 9,388,154 9,346,738; U.S. Published Patent Applications Nos. 2012-0197041, 2013-0331593, 2014-0024856, 2015-0299091, 2015-0376106, 2016-0107973, 2015-0315114, 2016-0152548, and 2016-0175319; PCT Publications No. WO2016/0055819 and WO2016/081658.

Various additional uses and/or forms of treprostinil are disclosed, for example, in U.S. Pat. Nos. 5,153,222, 5,234,953, 6,521,212, 6,756,033, 6,803,386, 7,199,157, 6,054,486, 7,417,070, 7,384,978, 7,879,909, 8,563,614, 8,252,839, 8,536,363, 8,410,169, 8,232,316, 8,609,728, 8,350,079, 8,349,892, 7,999,007, 8,658,694, 8,653,137, 9,029,607, 8,765,813, 9,050,311, 9,199,908, 9,278,901, 8,747,897, 9,358,240, 9,339,507, 9,255,064, 9,278,902, 9,278,903, 9,758,465; 9,422,223; 9,878,972; 9,624,156, 8,969,409, 10,716,793, 10,376,525; U.S. Published Patent Applications Nos. 2009-0036465, 2008-0200449, 2008-0280986, 2009-0124697, 2014-0275616, 2014-0275262, 2013-0184295, 2014-0323567, 2016-0030371, 2016-0051505, 2016-0030355, 2016-0143868, 2015-0328232, 2015-0148414, 2016-0045470, 2016-0129087, 2017-0095432; 2018-0153847; 2021-0330621 and PCT Publications Nos. WO00/57701, WO20160105538, WO2016038532, WO2018/058124; WO2021/211916.

A “prodrug” of treprostinil refers to compounds which are converted in vivo to treprostinil or its pharmaceutically active derivatives thereof, or to a compound described in PCT publication No. WO2005/007081; U.S. Pat. Nos. 7,384,978, 7,417,070, 7,544,713, 8,252,839, 8,410,169, 8,536,363, 9,050,311, 9,199,908, 9,278,901, 9,422,223; 9,624,156, 9,878,972, 9,371,264, 9,394,227, 9,505,737, 9,758,465, 9,643,911, 9,701,616, 9,776,982, 9,845,305, 9,957,200, 10,494,327, 10,053,414, 10,246,403, 10,344,012, 10,450,290, 10,464,877, 10,464,878, 10,703,706, 10,752,733, 9,255,064, 9,469,600, 10,010,518, 10,343,979, 10,526,274; U.S. Patent Application Publications Nos. 2018-0153847; 2021-0054009; 2021-0378996; U.S. patent application Ser. No. 17/549,573 filed Dec. 13, 2021; U.S. provisional patent application No. 63/156,110 filed Mar. 3, 2021, each of which is incorporated herein by reference in their entirety.

“Pharmaceutically acceptable salts” are physiologically acceptable salts of treprostinil, treprostinil prodrug or iloprost, as well as non-physiologically acceptable salts of treprostinil, treprostinil prodrug or iloprost. Pharmaceutically acceptable salts of treprostinil, treprostinil prodrug or iloprost are within the scope of the present technology and include base addition salts which retain the desired pharmacological activity and is not biologically undesirable (e.g., the salt is not unduly toxic, allergenic, or irritating, and is bioavailable). Treprostinil, treprostinil prodrug or iloprost has at least one acidic group, such as for example, a carboxylic acid group. Thus, treprostinil, treprostinil prodrug or iloprost can form a salt with a metal, such as an alkali metal or an alkali earth metal (e.g., Na⁺, Li⁺, K⁺, Ca²⁺, Mg²⁺, Zn²⁺), ammonia or an organic amine (e.g., dicyclohexylamine, trimethylamine, triethylamine, pyridine, picoline, ethanolamine, diethanolamine, triethanolamine, tromethamine) or basic amino acids (e.g., arginine, lysine, histidine and ornithine). Such salts may be prepared in situ during isolation and purification of the compounds or by separately reacting the purified compound in its free acid form with a suitable base, respectively, and isolating the salt thus formed.

The treatment can improve one or more physiological metrics associated with PH or alleviate or reduce one or more symptoms associated with PH. For example, in some embodiments, treatment can reduce the pulmonary arterial pressure. In other embodiments, treatment can result in improved exercise ability, such as improved distance on the six-minute walk test (6MWT). In some embodiments, treatment can result in alleviating or reducing one more symptoms of PH, such as decreasing shortness of breath or faintness.

In some embodiments, the combination of iloprost and treprostinil may be administered by inhalation for treating pulmonary arterial hypertension (PAH). PAH is characterized by a thickening (narrowing of the lumen) and stiffening of the pulmonary arteries. As a result, the right side of the heart may have to work harder to push blood through these narrowed arteries. This extra stress can cause the heart to lose its ability to pump enough blood through the lungs to meet the needs of the rest of the body.

In some embodiments, the combination of iloprost and treprostinil may be administered by inhalation for treating a pulmonary hypertension, WHO Group 3, i.e. a pulmonary hypertension due to a condition selected from a chronic lung disease and/or hypoxia (low oxygen levels). The Group 3 PH can be pulmonary hypertension associated with interstitial lung disease (PH-ILD).

The chronic lung disease may include an obstructive lung disease in which the lung airways are narrow and make it difficult to exhale, such as chronic obstructive pulmonary disease (COPD) and emphysema; a restrictive lung disease in which the lungs have a difficult time expanding when one inhales, such as interstitial lung disease or pulmonary fibrosis; sleep apnea; living in an area of high altitude for a long period of time; and various combinations of the above conditions.

In some embodiments, the chronic lung disease may include idiopathic interstitial pneumonia, such as idiopathic pulmonary fibrosis, idiopathic nonspecific interstitial pneumonia, respiratory bronchiolitis (e.g. respiratory bronchiolitis associated with interstitial lung disease), desquamative interstitial pneumonia, acute interstitial pneumonia; chronic hypersensitivity pneumonitis, occupational lung disease, pulmonary fibrosis, emphysema, connective tissue disease or any combination of the above conditions.

Treprostinil or its pharmaceutically acceptable salt and iloprost or its pharmaceutically acceptable salt may be administered in a therapeutically effect amount, which may be an amount effective to treat pulmonary hypertension.

While the present invention is not bound by any theory, treprostinil is effective against pulmonary hypertension (such as pulmonary arterial hypertension or pulmonary hypertension associated with interstitial lung disease) due at least in part to its beneficial vasodilatory effects on pulmonary vasculature and/or its ability to induce remodeling of the pulmonary vasculature. Iloprost has a shorter half-life than treprostinil. Iloprost also is generally administered more frequently because of its shorter half-life. However, iloprost has a more substantial inotropic effect than treprostinil, which can be beneficial to cardiac function. Combining treprostinil and/or iloprost's pulmonary vasculature effects with a relatively short lived but substantial inotropic boost on the right heart from iloprost may provide additional benefits for some pulmonary hypertension patients that would not be provided if either drug were administered as a mono-therapy. For example, the combination of treprostinil and iloprost may be administered pro re nata, (i.e., as needed) to a patient experiencing at least one unwanted symptom of pulmonary hypertension, such as difficulty breathing or shortness of breath. Iloprost in the combination may provide a relatively short lived but substantial inotropic boost, which may reduce or ameliorate the at least one unwanted symptom of pulmonary hypertension, such as difficulty breathing or shortness of breath.

In some embodiments, the iloprost administered by inhalation in combination with treprostinil may be iloprost as free acid or iloprost tromethamine. In some embodiments, the treprostinil administered by inhalation in combination with iloprost may be treprostinil as free acid or treprostinil sodium.

In some embodiments, the iloprost and treprostinil can be administered simultaneously. For example, a patient can be administered a both iloprost and treprostinil using any suitable delivery mechanism, such as a nebulizer or a dry powder inhaler. The simultaneous delivery can be administered using a composition comprising both treprostinil and iloprost. The relative amounts of treprostinil and iloprost can be selected by a skilled artisan based on the desired dosage, which is discussed in more detail below. In some embodiments, the simultaneous administration is achieved using a rescue inhaler-type device that delivers a fixed amount of drugs or imposes an upper limit on the dosage that can be delivered.

In other embodiments, the iloprost and treprostinil can be administered separately. These embodiments may be useful to adjust the relative dosages of the drugs. For example, iloprost can be administered preceded or followed by administration of treprostinil. The drugs can be administered using the same or different delivery mechanisms. The drugs can be administered using the same or different inhalation device. For example, in some embodiments, one drug can be administered using a nebulizer and the other using a dry powder inhaler. When iloprost and treprostinil are separately administered, the administrations can be immediate, e.g., administration of treprostinil immediately followed by administration of iloprost (or vice versa), or separated in time, e.g., administration of treprostinil followed by administration of iloprost (or vice versa). For example, the administration of iloprost and treprostinil can be separated by about 2 minutes, 5 minutes, 10 minutes, 30 minutes, 1 hour, 2 hours, 3 hours, or 4 hours. In some embodiments, the drugs are administered at differing frequencies. For example, treprostinil can be administered one, two, three, or four times a day, and iloprost can be administered four, five, six, or more than six times daily. In some embodiments, one of the drugs, such as iloprost, is administered pro re nata, and the other drug is administered based on a fixed dosage regimen, e.g., one, two, three or four times daily.

In some embodiments, administering of iloprost and treprostinil may be performed in a single administering event or in a single dose event. In some embodiments, a number of breaths in the single administering event or in the single dose event may not exceed 20 breaths (or inhalations) or 19 breaths (or inhalations) or 18 breaths (or inhalations) or 17 breaths (or inhalations) or 16 breaths (or inhalations) or 15 breaths (or inhalations) or 14 breaths (or inhalations) or 13 breaths (or inhalations) or 12 breaths (or inhalations) or 11 breaths (or inhalations) or 10 breaths (or inhalations) or 9 breaths (or breaths (or inhalations) inhalations) or 8 breaths (or inhalations) or 7 breaths (or inhalations) or 6 breaths (or inhalations) or 5 breaths (or inhalations) or 4 breaths (or inhalations) or 3 breaths (or inhalations) or 2 breaths (or inhalations) or 1 breath (or inhalation).

A number of single administering events per day for administering treprostinil, its prodrug, its pharmaceutically acceptable salt or a pharmaceutically acceptable salt of its prodrug administered by inhalation may vary. For example, the number of single administering events per day may be 1, 2, 3, 4, 5 or 6 per day. In some embodiments, the number of single administering events per day may be at least two, such as from 2 to 5 events per day or from 2 to 3 events per day.

WA A dose or amount of treprostinil, its prodrug, its pharmaceutically acceptable salt or a pharmaceutically acceptable salt of its prodrug administered by inhalation in a single administering event may vary. In some embodiments, the single administering event dose of treprostinil may be at least 5 μg or at least 6 μg. In some embodiments, the single administering event dose of treprostinil may be from 5 μg to 120 μg or from 18 μg to 120 or from 6 μg to 96 μg or from 7.5 μg to 100 μg or 10 μg to 100 μg or 15 μg to 100 μg from 15 μg to 90 μg or from 15 μg to 75 μg or from 30 μg to 75 μg or any value or subrange within these ranges.

A dose or amount of iloprost or its pharmaceutically acceptable salt administered by inhalation in a single administering event may vary. In some embodiments, the single administering event dose of iloprost may be at least 2 μg or at least 2.5 μg. In some embodiments, the single administering event dose of iloprost may be from 2 μg to 10 μg or from 2 μg to 9 μg or from 2.5 μg to 5 μg or any value or subrange within these ranges. In some embodiments, a daily dose of iloprost administered over all administering events of the single day may be less than 15 μg or no more or less than 14 μg or no more or less than 13 μg or no more or less than 12 μg or no more or less than 11 μg or no more or less than 10 μg or no more or less than 9 μg or no more or less than 8 μg or no more or less than 7 μg or no more or less than 6 μg or no more or less than 5 μg.

In some embodiments, iloprost and treprostinil may be administered by a single inhalation device, such as a nebulizer or a dry powder inhaler, which may contain two separate compositions, one containing iloprost and one containing treprostinil.

Yet in some embodiments, iloprost and treprostinil may be administered in a single composition, which comprises (a) iloprost or its pharmaceutically acceptable salt and (b) treprostinil, its prodrug, its pharmaceutically acceptable salt or a pharmaceutically acceptable salt of its prodrug. Although the present invention is not bound by any theory, the present inventors have discovered that treprostinil may exert a stabilizing effect when it is combined in a composition with iloprost compared to the stability of iloprost when it is not combined with treprostinil in the same composition. In some embodiments, such a single composition may be a solution comprising (a) iloprost or its pharmaceutically acceptable salt and (b) treprostinil, its prodrug, its pharmaceutically acceptable salt or a pharmaceutically acceptable salt of its prodrug. A concentration of treprostinil in such solution may vary. In some embodiments, the treprostinil concentration may be from 200 μg/ml to 2000 μg/ml or from 300 μg/ml to 1500 μg/ml or from 400 μg/ml to 1200 μg/ml or any value or subrange within these ranges. For example, in a certain embodiment, the treprostinil concentration may be 600 μg/ml. A concentration of iloprost in such solution may vary. In some embodiments, the iloprost concentration may be from 5 μg/ml to 50 μg/ml or from 5 μg/ml to 40 μg/ml or from 5 μg/ml to 30 μg/ml or any value or subrange within these ranges. For example, in a certain embodiment, the iloprost concentration may be 10 μg/ml or 20 μg/ml.

In some embodiments, the composition comprising (a) iloprost or its pharmaceutically acceptable salt and (b) treprostinil, its prodrug, its pharmaceutically acceptable salt or a pharmaceutically acceptable salt of its prodrug, may be administered by an oral inhalation or a nasal inhalation. In some embodiments, the composition comprising (a) iloprost or its pharmaceutically acceptable salt and (b) treprostinil, its prodrug, its pharmaceutically acceptable salt or a pharmaceutically acceptable salt of its prodrug, may be administered by an inhalation device, such as a nebulizer. In some embodiments, the composition comprising (a) iloprost or its pharmaceutically acceptable salt and (b) treprostinil, its prodrug, its pharmaceutically acceptable salt or a pharmaceutically acceptable salt of its prodrug, may be administered, for example, by a pulsed inhalation device, such as a metered dose inhaler and/or a pulsed nebulizer. Pulsed inhalation devices are disclosed, for example, in U.S. patent application publication No. 20080200449, U.S. Pat. Nos. 9,358,240; 9,339,507; 10,376,525; and 10,716,793, each of which is incorporated herein by reference in its entirety.

In some embodiments, the composition comprising (a) iloprost or its pharmaceutically acceptable salt and (b) treprostinil, its prodrug, its pharmaceutically acceptable salt or a pharmaceutically acceptable salt of its prodrug may be administered as a dry powder composition. The dry powder composition may be administered by a dry powder inhaler, which may be a pulsed dry powder inhaler. Dry powder inhalers are disclosed, for example, in U.S. Pat. Nos. 7,305,986, 7,464,706, 8,499,757 and 8,636,001, PCT publication WO2019237028, each of which is incorporated by reference.

In some embodiments, a dry powder inhaler may comprise a cartridge, which may be a replaceable cartridge, comprising the dry powder composition. In some embodiments, a dry powder inhaler may a breath-powered inhaler which may be compact, reusable or disposable. A dry powder inhaler may have a number of various shapes and sizes, and may comprise a system of airflow conduit pathways for the effective and rapid delivery of the powder medicament to the lungs and/or the systemic circulation.

In some embodiments, in addition to a) iloprost or its pharmaceutically acceptable salt and (b) treprostinil, its prodrug, its pharmaceutically acceptable salt or a pharmaceutically acceptable salt of its prodrug, the dry powder composition may further a diketopiperazine, such as (E)-3,6-bis[4-(N-carbonyl-2-propenyl)amidobutyl]-2,5-diketopiperazine (FDKP).

Patients treated using the methods described herein may be treated with other therapies. In some embodiments, subjects will be on background therapy for PH and add to that a combination of treprostinil and iloprost. For example, the combination of iloprost and treprostinil may be added to existing therapy to address acute symptoms, such as exercise-induced symptoms. Background therapies may include phosphodiesterase-5 inhibitors (e.g., sildenafil and tadalafil), soluble guanylate cyclase stimulators (sGCS) (e.g., riociguat), endothelin receptor antagonists (ERA) (e.g., bosentan and ambrisentan), or other prostacyclins.

In some embodiments, iloprost and treprostinil may be in a single pharmaceutical formulation. In some embodiments, the pharmaceutical formulation may be a liquid formulation. In some embodiments, the pharmaceutical formulation, such as a liquid pharmaceutical formulation, may be an inhalable pharmaceutical formulation, i.e. a formulation administered by inhalation.

A concentration of treprostinil in the pharmaceutical formulation, such as a liquid pharmaceutical formulation, may vary. In some embodiments, the treprostinil concentration may be from 200 μg/ml to 2000 μg/ml or from 300 μg/ml to 1500 μg/ml or from 400 μg/ml to 1200 μg/ml or any value or subrange within these ranges. For example, in a certain embodiment, the treprostinil concentration may be 600 μg/ml. A concentration of iloprost in in the pharmaceutical formulation, such as a liquid pharmaceutical formulation, may vary. In some embodiments, the iloprost concentration may be from 5 μg/ml to 50 μg/ml or from 5 μg/ml to 40 μg/ml or from 5 μg/ml to 30 μg/ml or any value or subrange within these ranges. For example, in a certain embodiment, the iloprost concentration may be 10 μg/ml or 20 μg/ml.

In some embodiments, in addition to iloprost and treprostinil, the pharmaceutical formulation, such as a liquid pharmaceutical formulation, may also include a buffer. In some embodiments, the buffer may be a phosphate buffer, such as sodium phosphate buffer. For example, in some embodiments, the formulation may include about 5-15 mM sodium phosphate buffer, more preferably about 9-11 mM sodium phosphate buffer, and most preferably about 10 mM sodium phosphate buffer.

In some embodiments, the pharmaceutical formulation, such as a liquid pharmaceutical formulation, may also include a salt. The salt may be, for example, a halide of an alkali metal, such as sodium chloride or sodium iodide. For example, in some embodiments, the formulation may include from about 20 mM to 1000 mM or from about 50 mM to about 500 mM or from about 100 mM to about 200 mM or from about 115 mM to about 125 mM.

In some embodiments, the pharmaceutical formulation, such as a liquid pharmaceutical formulation, may be an isotonic solution.

In some embodiments, the pharmaceutical formulation, such as a liquid pharmaceutical formulation, may have pH from about 5.8 to about 7.2 or from about 5.9 to about 7.1 or from about 6.0 to about 7.0 or from about 6.1 to 6.9 or from about 6.2 to about 6.8 or about 6.5.

In some embodiments, the pharmaceutical formulation, such as a liquid pharmaceutical formulation, may have an osmolality from about 200 mOsm/kg to about 500 mOsm/kg or from about 250 mOsm/kg to about 400 mOsm/kg or from about 270 mOsm/kg to about 340 mOsm/kg.

In some embodiments, the pharmaceutical formulation, such as a liquid pharmaceutical formulation, may include ilorpost per se or its pharmaceutically acceptable salt, such as a tromethamine salt. In some embodiments, the pharmaceutical formulation, such as a liquid pharmaceutical formulation, may include treprostinil as a free base or its pharmaceutically acceptable salt, such as a sodium salt.

The pharmaceutical formulation, such as a liquid pharmaceutical formulation, may be used for treating pulmonary hypertension by being administered to a subject, such as human being. In some embodiments, administering the pharmaceutical formulation, such as a liquid pharmaceutical formulation, may be performed by inhalation. In some embodiments, the pulmonary hypertension may be pulmonary arterial hypertension. Yet in some embodiments, pulmonary hypertension may be pulmonary hypertension associated with interstitial lung disease.

In some embodiments, administering the pharmaceutical formulation, such as a liquid pharmaceutical formulation, may be performed in a single administering event or in a single dose event. In some embodiments, a number of breaths in the single administering event or in the single dose event may not exceed 20 breaths (or inhalations) or 19 breaths (or inhalations) or 18 breaths (or inhalations) or 17 breaths (or inhalations) or 16 breaths (or inhalations) or 15 breaths (or inhalations) or 14 breaths (or inhalations) or 13 breaths (or inhalations) or 12 breaths (or inhalations) or 11 breaths (or inhalations) or 10 breaths (or inhalations) or 9 breaths (or breaths (or inhalations) inhalations) or 8 breaths (or inhalations) or 7 breaths (or inhalations) or 6 breaths (or inhalations) or 5 breaths (or inhalations) or 4 breaths (or inhalations) or 3 breaths (or inhalations) or 2 breaths (or inhalations) or 1 breath (or inhalation).

In some embodiments, a single event of administering the pharmaceutical formulation, such as a liquid pharmaceutical formulation, may be less than about 10, 7, 5, 3, 2, or 1 minutes.

A number of single administering events per day for administering the pharmaceutical formulation, such as a liquid pharmaceutical formulation, by inhalation may vary. For example, the number of single administering events per day may be 1, 2, 3, 4, 5 or 6 per day. In some embodiments, the number of single administering events per day may be at least two, such as from 2 to 5 events per day or from 2 to 3 events per day.

In some embodiments, the pharmaceutical formulation, such as a liquid pharmaceutical formulation, may be administered, for example, by a pulsed inhalation device, such as a metered dose inhaler and/or a pulsed nebulizer. Pulsed inhalation devices are disclosed, for example, in U.S. patent application publication No. 20080200449, U.S. Pat. Nos. 9,358,240; 9,339,507; 10,376,525; and 10,716,793, each of which is incorporated herein by reference in its entirety.

A dose or amount of iloprost or its pharmaceutically acceptable salt administered by inhalation in a single administering event of the pharmaceutical formulation, such as a liquid pharmaceutical formulation, may vary. In some embodiments, the single administering event dose of iloprost may be at least 2 μg or at least 2.5 μg. In some embodiments, the single administering event dose of iloprost may be from 2 μg to 10 μg or from 2 μg to 9 μg or from 2.5 μg to 5 μg or any value or subrange within these ranges. In some embodiments, a daily dose of iloprost administered over all administering events of the single day may be less than 15 μg or no more or less than 14 μg or no more or less than 13 μg or no more or less than 12 μg or no more or less than 11 μg or no more or less than 10 μg or no more or less than 9 μg or no more or less than 8 μg or no more or less than 7 μg or no more or less than 6 μg or no more or less than 5 μg.

A dose or amount of treprostinil, its prodrug, its pharmaceutically acceptable salt or a pharmaceutically acceptable salt of its prodrug administered by inhalation in a single administering event of the pharmaceutical formulation, such as a liquid pharmaceutical formulation, may vary. In some embodiments, the single administering event dose of treprostinil may be at least 5 μg or at least 6 μg. In some embodiments, the single administering event dose of treprostinil may be from 5 μg to 120 μg or from 18 μg to 120 μg or from 6 μg to 96 μg or from 7.5 μg to 100 μg or 10 μg to 100 μg or 15 μg to 100 μg from 15 μg to 90 μg or from 15 μg to 75 μg or from 30 μg to 75 μg or any value or subrange within these ranges.

In some embodiments, the pharmaceutical formulation, such as a liquid pharmaceutical formulation, may be administered pro re nata, (i.e., as needed) to a patient experiencing at least one unwanted symptom of pulmonary hypertension, such as difficulty breathing or shortness of breath.

The pharmaceutical formulation, such as a liquid pharmaceutical formulation, containing both iloprost and treprostinil may have a higher stability of iloprost than that of other-wise identical iloprost formulations that do not contain treprostinil.

The pharmaceutical formulation, such as a liquid pharmaceutical formulation, containing both iloprost and treprostinil may be such that the formulation, which may be freshly prepared formulation, is storage stable. Preferably, the formulation is capable of being stored or is stored for a storage period of at least 18 months, more preferably at least 24 months. In some embodiments, the formulation is capable of being stored or is stored for at least two weeks or at least one month or at least 6 weeks or at least two months or at least 10 weeks or at least three months or from 2 weeks to three months or from one month to three months or one month to two months. In some embodiments, preferably an amount of iloprost in the formulation after the storage would be at least 90% or at least 91% or at least 92% or at least 93% or at least 94% of an amount of iloprost in the formulation before the storage. In some embodiments, such storage may be performed without cooling the formulation below a temperature such as about 20 C. For example, in some embodiments, the storage may be performed at a temperature from about 20 C to about 50 C or from about 22 C to about to about 45 C or from about 25 C to about 40 C.

In some embodiments, the pharmaceutical formulation, such as a liquid pharmaceutical formulation, may be stored in a container. In some embodiments, the container may be a sealed container. In some embodiments, the container may be a vial or an ampule. In some embodiments, the container may be a glass container, i.e. a container made of glass. In some embodiments, the container may be a plastic container, i.e. a container made of plastic. Non-limiting examples of plastics include polyolefins, such as polypropylene and polyethylene, such as low density polyethylene (LDPE). In some embodiments, the container is fitted with a removeable cap that can be opened and closed, for example a rubber sealed cap that attaches to a glass container.

Also are provide are dosage forms of the pharmaceutical formulation, such as a liquid pharmaceutical formulation, in a container. In some embodiments, the dosage form may be a dosage form for inhalation. In some embodiments, the container may be a sealed container. In some embodiments, the container may be a vial or an ampule. In some embodiments, the container may be a glass container, i.e. a container made of glass. In some embodiments, the container may be a plastic container, i.e. a container made of plastic. Non-limiting examples of plastics include polyolefins, such as polypropylene and polyethylene, such as low density polyethylene (LDPE). An amount of the pharmaceutical formulation, such as a liquid pharmaceutical formulation, in the container may vary. In some embodiments, the container may contain from 0.5 ml to 50 ml or from 1 ml to 30 ml or from 2 ml to 20 ml of the liquid pharmaceutical formulation.

Embodiments described herein are further illustrated by, though in no way limited to, the following working examples.

### Example 1

### Iloprost and Treprostinil Formulation Development

The purpose of this Example is to describe the formulation development work for an inhalation formulation of Iloprost and Treprostinil. An isotonic formulation was developed targeting a pH of 6.5.

TABLE 1
DEFINITIONS

| Term | Definition |
|---|---|
| ACN | Acetonitrile |
| HPLC | High Performance Liquid Chromatography |
| H₃PO₄ | Phosphoric Acid |
| NaCl | Sodium Chloride |
| UV | Ultraviolet |

### Procedure

### Solubility Evaluation

The solubility of iloprost at 100 μg/mL was evaluated at five different pH levels by visual inspection and HPLC-UV analysis. A stock solution of Iloprost was prepared in ethanol at 1 mg/mL. The stock solution was diluted 10-fold in each buffer and mixed well. The buffered solutions were visually inspected for any precipitation. The buffered solutions were filtered with 0.45 μm nylon filters prior to analysis by HPLC. The buffers and associated pH are shown in Table 2. HPLC conditions for solubility analysis are shown in Table 3.

TABLE 2
Buffers and associated pH.

| Buffer | Measured pH |
|---|---|
| 10 mM Citrate | 2.89 |
| 10 mM Acetate | 5 |
| 10 mM Phosphate | 6.23 |
| 10 mM Phosphate | 7.27 |
| 10 mM Phosphate | 8.60 |

TABLE 3
HPLC conditions for solubility analysis.

| | % A (0.1% | % B (0.1% |
|---|---|---|
| Time (minutes) | H₃PO₄ in Water) | H₃PO₄ in ACN) |
| 0 | 80 | 20 |
| 5 | 80 | 20 |
| 15 | 50 | 50 |
| 25 | 20 | 80 |
| 25.1 | 80 | 20 |
| 30 | 80 | 20 |

| | |
|---|---|
| Flow Rate | 0.4 mL/min |
| Column | Waters BEH HPLC C8 (1.7 μm, 2.1 mm × 100 mm) |
| Column | 40° C. |
| Compartment | |
| Injection Volume | 2 μL |
| Collection and Rate | 205 nm, 5 Hz |
| Needle Wash | 50:50 Water:ACN |

### Formulation Development

### Iloprost Formulation Development

Iloprost formulations were prepared at 10 μg/mL with two different NaCl concentrations. A stock Iloprost solution of 5 mg/mL was prepared in ethanol and diluted 500-fold in the formulations. The formulations were buffered with a 10 mM sodium phosphate buffer at pH 6.5. NaCl was added for a final salt concentration of 115 mM and 125 mM. The osmolality of the formulations was evaluated.

### Iloprost and Treprostinil Formulation Development

Two formulations of Iloprost and Treprostinil were prepared at 10 μg/mL Iloprost and 600 μg/mL, Treprostinil. A stock Iloprost solution of 5 mg/mL was prepared in ethanol and diluted 500-fold in the formulations. Treprostinil was quantitatively weighed into the formulations. The formulations were buffered with a 10 mM sodium phosphate buffer at two different NaCl concentrations, 115 and 125 mM. Following the addition of Treprostinil, 3-4 drops of 1N NaOH were added to solubilize the Treprostinil in the buffer. Further pH adjustment was done to bring the formulation pH within 0.5 of the 6.5 target. The osmolality of the formulations was evaluated.

Two formulations of Iloprost and Treprostinil was prepared at 20 μg/mL Iloprost and 600 μg/mL, Treprostinil. A stock Iloprost solution of 5 μg/mL was prepared in ethanol and diluted 250-fold in the formulations. Treprostinil was quantitatively weighed into the formulations. The formulations were buffered with a 10 mM sodium phosphate buffer at two different NaCl concentrations, 115 and 125 mM. Following the addition of Treprostinil, 3-4 drops of 1N NaOH were added to solubilize the Treprostinil in the buffer. Further pH adjustment was done to bring the formulation pH within 0.5 of the 6.5 target. The osmolality of the formulations was evaluated.

### Results and Discussion

### Solubility Evaluation

No visual solubility issues were noted. Visual observations and pH are shown in Table 4. Chromatograms of the five different buffer solutions containing 100 μg/mL of Iloprost are shown in the FIGURE. Although no visual particles were noted, the chromatographic analysis indicates potential solubility issues below pH 6.

TABLE 4
Physical observations and pH of buffered Iloprost solutions.

| | Initial | Final | |
|---|---|---|---|
| Buffer | pH | pH | Observation |
| 10 mM Citrate | 2.89 | 2.99 | Colorless, no visible particulate matter |
| 10 mM Acetate | 5.00 | 5.08 | Colorless, no visible particulate matter |
| 10 mM Phosphate | 6.23 | 6.36 | Colorless, no visible particulate matter |
| 10 mM Phosphate | 7.27 | 7.42 | Colorless, no visible particulate matter |
| 10 mM Phosphate | 8.60 | 8.59 | Colorless, no visible particulate matter |

### Formulation Development

### Iloprost Formulation Development

Both the 115 and 125 mM salt concentrations were within the isotonic range. The 125 mM formulation was closer to the target of 290 mOsm/kg. The formulations and osmolality are summarized in Table 5.

TABLE 5
Iloprost formulations and osmolality.

| | Measured | Measured Osmolality |
|---|---|---|
| Formulation | pH | (mOsm/kg) |
| 10 μg/mL Iloprost, | 6.45 | 272 |
| pH 6.5, 115 mM NaCl | | |
| 10 μg/mL Iloprost, | 6.45 | 290 |
| pH 6.5, 125 mM NaCl | | |

### Iloprost and Treprostinil Formulation Development

Treprostinil required the addition of base to solubilize in the formulation buffer. The additional pH adjustment causes an increase in the ions present. The 125 mM salt level in the second formulation (20 μg/mL Iloprost and 600 μg/mL Treprostinil) in combination with additional pH adjustment results in osmolality near the upper isotonic range. The 115 mM salt level is best suited to keep the formulations toward the center of the isotonic solution range. The formulations and osmolality are summarized in Table 6.

TABLE 6
Iloprost and Treprostinil formulations and osmolality.

| | | |
|---|---|---|
| | Measured | Measured Osmolality |
| Formulation | pH | (mOsm/kg) |

10 μg/mL Iloprost, 600 μg/mL Treprostinil, pH 6.5

| | | |
|---|---|---|
| 115 mM NaCl | 6.51 | 281 |
| 125 mM NaCl | 6.52 | 299 |

20 μg/mL Iloprost, 600 μg/mL Treprostinil, pH 6.5

| | | |
|---|---|---|
| 115 mM NaCl | 6.51 | 314 |
| 125 mM NaCl | 6.54 | 337 |

### Conclusion

A formulation for Iloprost at 10 μg/mL in a 10 mM sodium phosphate buffer with 125 mM NaCl at pH 6.5 was developed. Two formulations containing Iloprost (10 and 20 μg/mL) and Treprostinil at 600 μg/mL, in a 10 mM sodium phosphate buffer with 115 mM NaCl at pH 6.5 were developed.

### Example 2

### Stability Testing of Iloprost Formulation

TABLE 7
Stability Testing Summary for 10 μg/mL Iloprost, pH 6.5, 125 mM NaCl
at 25 ± 2° C./60 ± 5% RH, 5 mL glass vial, sample orientation: inverted

| Attribute | Testing Interval (Months) | | | | | |
|---|---|---|---|---|---|---|
| (Test Method) | T = 0 | | 1 | | 2 | 3 |
| Appearance | Clear, colorless | | Clear, colorless | | Clear, colorless | |
| | solution, essentially | | solution, essentially | | solution, essentially | |
| | free from visible | | free from visible | | free from visible | |
| | particulate matter. | | particulate matter. | | particulate matter. | |
| pH USP <791> | 6.6 | | 6.5 | | 6.6 | |
| Osmolality (mOsm/kg) | 288 | mOsm/kg | 290 | mOsm/kg | 289 mOsm/kg | |
| USP <785> | | | | | | |
| Iloprost Assay (% LC) | 93.6% | LC | 92.7% | LC | 75.1¹ | |
| “Fit for Purpose” | | | | | | |

¹Result confirmed with reinjection and revial.

TABLE 8
Individual Impurities Summary for 10 μg/mL Iloprost, pH 6.5, 125 mM
NaCl at 25 ± 2° C./60 ± 5% RH, 5 mL glass vial

| | |
|---|---|
| Relative | Interval (Months) |

| Retention Time | T = 0 | 1 | 2 | 3 |
|---|---|---|---|---|
| RRT~0.059-0.062 | ND | 1.4 | 0.99 | |
| RRT~0.067 | ND | ND | 0.59 | |
| RRT~0.214-0.220 | 0.53 | 0.51 | 0.55 | |
| RRT~0.418 | ND | 0.38 | ND | |
| RRT~0.433 | ND | ND | 0.25 | |
| RRT~0.438 | ND | ND | 0.21 | |
| RRT~0.450 | ND | ND | 0.16 | |
| RRT~0.535 | ND | ND | 1.2 | |
| RRT~0.543 | ND | ND | 0.90 | |
| RRT~0.576 | ND | ND | 0.44 | |
| RRT~0.584 | ND | ND | 0.38 | |
| RRT~0.590 | ND | ND | 0.57 | |
| RRT~0.653 | ND | ND | 0.16 | |
| RRT~0.672 | ND | ND | 0.12 | |
| RRT~0.707 | ND | ND | 0.32 | |
| RRT~0.724 | ND | ND | 0.28 | |
| RRT~0.772 | ND | ND | 1.2 | |
| RRT~0.795 | ND | ND | 0.97 | |
| Total Unspecified | 0.53 | 2.3 | 9.3 | |
| Impurities | | | | |

Report RRT and % adjusted area for all impurities ≥0.05% adjusted area
ND = Not Detected (<LOD). LOD stand for “Limit Of Detection”.
NR = Not Reported (≥LOD and <0.05%)
NT = Not Tested

TABLE 9
Stability Testing Summary for 10 μg/mL Iloprost, pH 6.5, 125 mM NaCl
at 40 ± 2° C./75 ± 5% RH, 5 mL glass vial, sample orientation: inverted

| Attribute | Testing Interval (Months) | | | | | | |
|---|---|---|---|---|---|---|---|
| (Test Method) | T = 0 | | 1 | | 2 | | 3 |
| Appearance | Clear, colorless | | Clear, colorless | | Clear, colorless | | |
| | solution, essentially | | solution, essentially | | solution, essentially | | |
| | free from visible | | free from visible | | free from visible | | |
| | particulate matter. | | particulate matter. | | particulate matter. | | |
| pH USP <791> | 6.6 | | 6.6 | | 6.6 | | |
| Osmolality (mOsm/kg) | 288 | mOsm/kg | 289 | mOsm/kg | 292 | mOsm/kg | |
| USP <785> | | | | | | | |
| Iloprost Assay (% LC) | 93.6% | LC | 92.9% | LC | 88.0% | LC | |
| “Fit for Purpose” | | | | | | | |

TABLE 10
Individual Impurities Summary for 10 μg/mL Iloprost, pH 6.5, 125 mM
NaCl at 40 ± 2° C./75 ± 5% RH, 5 mL glass vial

| | |
|---|---|
| Relative | Interval (Months) |

| Retention Time | T = 0 | 1 | 2 | 3 |
|---|---|---|---|---|
| RRT~0.059-0.062 | ND | 0.99 | 0.86 | |
| RRT~0.158 | ND | ND | 0.20 | |
| RRT~0.214-0.220 | 0.53 | 0.56 | 0.54 | |
| RRT~0.418 | ND | 0.45 | ND | |
| RRT~0.435 | ND | ND | 0.45 | |
| RRT~0.560 | ND | 0.19 | ND | |
| RRT~0.584 | ND | ND | 0.76 | |
| RRT~0.743 | ND | ND | 0.32 | |
| Total Unspecified | 0.53 | 2.2 | 3.1 | |
| Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 11
Stability Testing Summary for 10 μg/mL Iloprost, pH 6.5, 125 mM NaCl
at 25 ± 2° C./60 ± 5% RH, 3 mL LDPE ampules

| Attribute | Testing Interval (Months) | | | | | | |
|---|---|---|---|---|---|---|---|
| (Test Method) | T = 0 | | 1 | | 2 | | 3 |
| Appearance | Clear, colorless | | Clear, colorless | | Clear, colorless | | |
| | solution, essentially | | solution, essentially | | solution, essentially | | |
| | free from visible | | free from visible | | free from visible | | |
| | particulate matter. | | particulate matter. | | particulate matter. | | |
| pH USP <791> | 6.6 | | 6.6 | | 6.6 | | |
| Osmolality (mOsm/kg) | 288 | mOsm/kg | 291 | mOsm/kg | 286 | mOsm/kg | |
| USP <785> | | | | | | | |
| Iloprost Assay (% LC) | 93.6% | LC | 89.2% | LC¹ | 84.0% | LC | |
| “Fit for Purpose” | | | | | | | |

¹The two injections of the sample were ~4% different.

TABLE 12
Individual Impurities Summary for 10 μg/mL
Iloprost, pH 6.5, 125 mM NaCl at 25 ± 2°
C./60 ± 5% RH, 3 mL LDPE ampules

| | |
|---|---|
| Relative | Interval (Months) |

| Retention Time | T = 0 | 1 | 2 | 3 |
|---|---|---|---|---|
| RRT~0.059-0.062 | ND | 1.4 | 0.94 | |
| RRT~0.067 | ND | ND | 1.2 | |
| RRT~0.128 | ND | 0.4 | 0.48 | |
| RRT~0.214-0.220 | 0.53 | 0.5 | ND | |
| RRT~0.418 | ND | 0.67 | ND | |
| RRT~0.435 | ND | ND | 0.16 | |
| RRT~0.454-0.457 | ND | 0.13 | 0.12 | |
| RRT~0.462 | ND | 0.5 | ND | |
| RRT~0.500 | ND | 0.53 | ND | |
| RRT~0.535 | ND | ND | 0.25 | |
| RRT~0.543 | ND | ND | 0.20 | |
| RRT~0.576 | ND | ND | 0.16 | |
| RRT~0.590 | ND | ND | 0.14 | |
| RRT~0.623 | ND | 0.27 | ND | |
| RRT~0.634 | ND | ND | 0.29 | |
| RRT~0.654 | ND | 0.4 | ND | |
| RRT~0.724 | ND | ND | 0.13 | |
| RRT~0.772 | ND | ND | 0.13 | |
| RRT~0.795 | ND | ND | 0.19 | |
| RRT~0.820 | ND | ND | 0.41 | |
| RRT~0.877 | ND | 0.32 | ND | |
| RRT~0.939 | ND | 0.48 | ND | |
| I RRT~1.088 | ND | ND | 0.17 | |
| RRT~1.210 | ND | 0.93 | ND | |
| Total Unspecified | 0.53 | 6.5 | 4.9 | |
| Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 13
Stability Testing Summary for 10 μg/mL Iloprost, pH 6.5, 125 mM NaCl
at 40 ± 2° C./75 ± 5% RH, 3 mL LDPE ampules

| Attribute | Testing Interval (Months) | | | | | | |
|---|---|---|---|---|---|---|---|
| (Test Method) | T = 0 | | 1 | | 2 | | 3 |
| Appearance | Clear, colorless | | Clear, colorless | | Clear, colorless | | |
| | solution, essentially | | solution, essentially | | solution, essentially | | |
| | free from visible | | free from visible | | free from visible | | |
| | particulate matter. | | particulate matter. | | particulate matter. | | |
| pH USP <791> | 6.6 | | 6.6 | | 6.6 | | |
| Osmolality (mOsm/kg) | 288 | mOsm/kg | 289 | mOsm/kg | 284 | mOsm/kg | |
| USP <785> | | | | | | | |
| Iloprost Assay (% LC) | 93.6% | LC | 92.4% | LC | 88.7% | LC | |
| “Fit for Purpose” | | | | | | | |

TABLE 14
Individual Impurities Summary for 10 μg/mL
Iloprost, pH 6.5, 125 mM NaCl at 40 ± 2°
C./75 ± 5% RH, 3 mL LDPE ampules

| | |
|---|---|
| Relative | Interval (Months) |

| Retention Time | T = 0 | 1 | 2 | 3 |
|---|---|---|---|---|
| RRT~0.059-0.062 | ND | 0.92 | 0.78 | |
| RRT~0.122 | ND | 0.13 | ND | |
| RRT~0.127-0.128 | ND | 0.18 | 0.24 | |
| RRT~0.214-0.220 | 0.53 | 0.52 | 0.52 | |
| RRT~0.362 | ND | 0.13 | ND | |
| RRT~0.418 | ND | 0.88 | ND | |
| RRT~0.435 | ND | ND | 1.0 | |
| RRT~0.445 | ND | 0.51 | ND | |
| RRT~0.450 | ND | ND | 0.17 | |
| RRT~0.457 | ND | ND | 0.69 | |
| RRT~0.462 | ND | 0.63 | ND | |
| RRT~0.471 | ND | 0.38 | ND | |
| RRT~0.499-0.500 | ND | 0.43 | 0.22 | |
| RRT~0.623 | ND | 0.11 | ND | |
| RRT~0.654 | ND | 0.38 | ND | |
| RRT~0.692 | ND | 0.18 | ND | |
| RRT~0.707 | ND | ND | 0.26 | |
| RRT~0.724 | ND | ND | 0.13 | |
| RRT~1.119 | ND | 0.62 | ND | |
| RRT~1.367 | ND | 0.33 | ND | |
| Total Unspecified | 0.53 | 6.3 | 4.0 | |
| Impurities | | | | |

Report RRT and % adjusted area for all impurities ≥0.05% adjusted area
ND = Not Detected (<LOD)
NR = Not Reported (≥LOD and <0.05%)
NT = Not Tested

### Example 3

### Stability Testing of Iloprost/Treprostinil Formulation 1

TABLE 15
Stability Testing Summary for 10 μg/mL Iloprost, 600 μg/mL Treprostinil, pH
6.5, 115 mM NaCl at 25 ± 2° C./60 ± 5% RH, 5 mL glass vial. Sample orientation inverted

| Attribute | Testing Interval (Months) | | | | | | |
|---|---|---|---|---|---|---|---|
| (Test Method) | T = 0 | | 1 | | 2 | | 3 |
| Appearance | Clear, Colorless | | Clear, Colorless | | Clear, Colorless | | |
| | solution, essentially | | solution, essentially | | solution, essentially | | |
| | free from visible | | free from visible | | free from visible | | |
| | particulate matter. | | particulate matter. | | particulate matter. | | |
| pH USP <791> | 6.6 | | 6.6 | | 6.4 | | |
| Osmolality (mOsm/kg) | 280 | mOsm/kg | 281 | mOsm/kg | 280 | mOsm/kg | |
| USP <785> | | | | | | | |
| Iloprost Assay (% LC) | 96.0% | LC | 97.2% | LC | 92.2% | LC | |
| “Fit for Purpose” | | | | | | | |
| Treprostinil Assay (% LC) | 97.6% | LC | 96.7% | LC | 97.9% | LC | |
| “Fit for Purpose” | | | | | | | |

TABLE 16A
Iloprost Impurities Summary for 10 μg/mL Iloprost,
600 μg/mL Treprostinil, pH 6.5, 115 mM NaCl
at 25 ± 2° C./60 ± 5% RH, 5 mL glass vial

| | |
|---|---|
| Relative | Interval (Months) |

| Retention Time | T = 0 | 1 | 2 | 3 |
|---|---|---|---|---|
| RRT~0.059-0.062 | ND | 1.3 | 3.1 | |
| RRT~0.067 | ND | ND | 3.7 | |
| RRT~0.214-0.220 | ND | 0.14 | 0.11 | |
| RRT~0.418 | ND | 0.39 | ND | |
| RRT~0.462 | ND | 0.11 | ND | |
| RRT~0.584 | ND | ND | 0.38 | |
| RRT~0.820 | ND | ND | 0.10 | |
| Total Iloprost | 0.00 | 1.9 | 7.4 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 16B
Treprostinil Impurities Summary for 10 μg/mL
Iloprost, 600 μg/mL Treprostinil, pH 6.5, 115
mM NaCl at 25 ± 2° C./60 ± 5% RH, 5 mL glass vial

| | |
|---|---|
| Relative | Interval (Months) |

| Retention Time | T = 0 | 1 | 2 | 3 |
|---|---|---|---|---|
| T RRT~1.0.17 | ND | ND | 0.10 | |
| T RRT~1.308 | 0.18 | 0.11 | ND | |
| T RRT~1.684 | 0.09 | 0.06 | ND | |
| T RRT~1.757 | ND | ND | ND | |
| Total Treprostinil | 0.27 | 0.17 | 0.10 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 17
Stability Testing Summary for 10 μg/mL Iloprost, 600 μg/mL Treprostinil, pH
6.5, 115 mM NaCl at 40 ± 2° C./75 ± 5% RH, 5 mL glass vial Sample Orientation: Inverted

| Attribute | Testing Interval (Months) | | | | | | |
|---|---|---|---|---|---|---|---|
| (Test Method) | T = 0 | | 1 | | 2 | | 3 |
| Appearance | Clear, Colorless | | Clear, Colorless | | Clear, Colorless | | |
| | solution, essentially | | solution, essentially | | solution, essentially | | |
| | free from visible | | free from visible | | free from visible | | |
| | particulate matter. | | particulate matter. | | particulate matter. | | |
| pH USP <791> | 6.6 | | 6.6 | | 6.6 | | |
| Osmolality (mOsm/kg) | 280 | mOsm/kg | 280 | mOsm/kg | 280 | mOsm/kg | |
| USP <785> | | | | | | | |
| Iloprost Assay (% LC) | 96.0% | LC | 96.8% | LC | 93.2% | LC | |
| “Fit for Purpose” | | | | | | | |
| Treprostinil Assay (% LC) | 97.6% | LC | 97.0% | LC | 97.9% | LC | |
| “Fit for Purpose” | | | | | | | |

TABLE 18A
Iloprost Impurities Summary for 10 μg/mL Iloprost,
600 μg/mL Treprostinil, pH 6.5, 115 mM NaCl
at 40 ± 2° C./75 ± 5% RH, 5 mL glass vial

| | |
|---|---|
| Relative | Interval (Months) |

| Retention Time | T = 0 | 1 | 2 | 3 |
|---|---|---|---|---|
| RRT~0.059-0.062 | ND | 1.2 | 0.97 | |
| RRT~0.158 | ND | ND | 0.16 | |
| RRT~0.214-0.220 | ND | 0.12 | 0.13 | |
| RRT~0.418 | ND | 0.57 | ND | |
| RRT~0.435 | ND | ND | 0.46 | |
| RRT~0.560 | ND | 0.21 | ND | |
| RRT~0.584 | ND | ND | 0.66 | |
| RRT~0.743 | ND | ND | 0.37 | |
| Total Iloprost | 0.00 | 2.1 | 2.7 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 18B
Treprostinil Impurities Summary for 10 μg/mL
Iloprost, 600 μg/mL Treprostinil, pH 6.5, 115
mM NaCl at 40 ± 2° C./75 ± 5% RH, 5 mL glass vial

| | |
|---|---|
| Relative | Interval (Months) |

| Retention Time | T = 0 | 1 | 2 | 3 |
|---|---|---|---|---|
| T RRT~1.017 | ND | ND | 0.10 | |
| T RRT~1.308 | 0.18 | ND | ND | |
| T RRT~1.684 | 0.09 | 0.06 | 0.06 | |
| T RRT~1.757 | ND | ND | ND | |
| Total Treprostinil | 0.27 | 0.06 | 0.16 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 19
Stability Testing Summary for 10 μg/mL Iloprost, 600 μg/mL Treprostinil,
pH 6.5, 115 mM NaCl at 25 ± 2° C./60 ± 5% RH, 3 mL LDPE ampules

| Attribute | Testing Interval (Months) | | | | | | |
|---|---|---|---|---|---|---|---|
| (Test Method) | T = 0 | | 1 | | 2 | | 3 |
| Appearance | Clear, Colorless | | Clear, Colorless | | Clear, Colorless | | |
| | solution, essentially | | solution, essentially | | solution, essentially | | |
| | free from visible | | free from visible | | free from visible | | |
| | particulate matter. | | particulate matter. | | particulate matter. | | |
| pH USP <791> | 6.6 | | 6.6 | | 6.6 | | |
| Osmolality (mOsm/kg) | 280 | mOsm/kg | 280 | mOsm/kg | 279 | mOsm/kg | |
| USP <785> | | | | | | | |
| Iloprost Assay (% LC) | 96.0% | LC | 96.9% | LC | 93.8% | LC | |
| “Fit for Purpose” | | | | | | | |
| Treprostinil Assay (% LC) | 97.6% | LC | 96.9% | LC | 98.0% | LC | |
| “Fit for Purpose” | | | | | | | |

TABLE 20A
Iloprost Impurities Summary for 10 μg/mL Iloprost,
600 μg/mL Treprostinil, pH 6.5, 115 mM NaCl
at 25 ± 2° C./60 ± 5% RH, 3 mL LDPE ampules

| | |
|---|---|
| Relative | Interval (Months) |

| Retention Time | T = 0 | 1 | 2 | 3 |
|---|---|---|---|---|
| RRT~0.059-0.062 | ND | 1.7 | 0.90 | |
| RRT~0.128 | ND | 0.16 | ND | |
| RRT~0.214-0.220 | ND | 0.12 | 0.11 | |
| RRT~0.418 | ND | 0.6 | ND | |
| RRT~0.435 | ND | ND | 0.56 | |
| RRT~0.445 | ND | 0.1 | ND | |
| RRT~0.454 | ND | 0.51 | ND | |
| RRT~0.457-0.462 | ND | 0.16 | 0.12 | |
| RRT~0.499-0.500 | ND | ND | 0.13 | |
| RRT~0.654 | ND | 0.16 | ND | |
| RRT~0.820 | ND | ND | 0.23 | |
| Total Iloprost | 0 | 3.5 | 2.1 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 20B
Treprostinil Impurities Summary for 10 μg/mL
Iloprost, 600 μg/mL Treprostinil, pH 6.5, 115
mM NaCl at 25 ± 2° C./60 ± 5% RH, 3 mL LDPE ampules

| | |
|---|---|
| Relative | Interval (Months) |

| Retention Time | T = 0 | 1 | 2 | 3 |
|---|---|---|---|---|
| T RRT~1.017 | ND | ND | 0.10 | |
| T RRT~1.308 | 0.18 | 0.09 | 0.06 | |
| T RRT~1.684 | 0.09 | ND | ND | |
| T RRT~1.757 | ND | ND | ND | |
| Total Treprostinil | 0.27 | 0.09 | 0.16 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 21
Stability Testing Summary for 10 μg/mL Iloprost, 600 μg/mL Treprostinil,
pH 6.5, 115 mM NaCl at 40 ± 2° C./75 ± 5% RH, 3 mL LDPE ampules

| | Testing Interval (Months) | | | | | | |
|---|---|---|---|---|---|---|---|
| Attribute (Test Method) | T = 0 | | 1 | | 2 | | 3 |
| Appearance | Clear, Colorless | | Clear, Colorless | | Clear, Colorless | | |
| | solution, essentially | | solution, essentially | | solution, essentially | | |
| | free from visible | | free from visible | | free from visible | | |
| | particulate matter. | | particulate matter. | | particulate matter. | | |
| pH USP <791> | 6.6 | | 6.6 | | 6.7 | | |
| Osmolality USP <785> | 280 | mOsm/kg | 279 | mOsm/kg | 276 | mOsm/kg | |
| Iloprost Assay (% LC) | 96.0% | LC | 97.7% | LC | 93.4% | LC | |
| “Fit for Purpose” | | | | | | | |
| Treprostinil Assay (% LC) | 97.6% | LC | 97.5% | LC | 98.5% | LC | |
| “Fit for Purpose” | | | | | | | |

TABLE 22A
Iloprost Impurities Summary for 10 μg/mL Iloprost,
600 μg/mL Treprostinil, pH 6.5, 115 mM NaCl
at 40 ± 2° C./75 ± 5% RH, 3 mL L.DPE ampules

Interval (Months)

| Relative Retention Time | T = 0 | 1 | 2 | 3 |
|---|---|---|---|---|
| RRT~0.059-0.062 | ND | 1.4 | 0.90 | |
| RRT~0.078 | ND | 0.14 | ND | |
| RRT~0.122-0.127 | ND | 0.16 | 0.24 | |
| RRT~0.128 | ND | 0.11 | ND | |
| RRT~0.214-0.220 | ND | 0.15 | 0.12 | |
| RRT~0.405 | ND | 0.12 | ND | |
| RRT~0.418 | ND | 0.48 | ND | |
| RRT~0.435 | ND | ND | 1.2 | |
| RRT~0.445 | ND | 0.48 | ND | |
| RRT~0.454-0.457 | ND | 0.1 | 0.66 | |
| RRT~0.462 | ND | 0.41 | ND | |
| RRT~0.471 | ND | 0.43 | ND | |
| RRT~0.500 | ND | 0.14 | ND | |
| RRT~0.654 | ND | 0.17 | ND | |
| RRT~0.672 | ND | ND | 0.11 | |
| RRT~0.692 | ND | 0.24 | ND | |
| RRT~0.707 | ND | ND | 0.24 | |
| RRT~0.724 | ND | ND | 0.15 | |
| RRT~0.820 | ND | ND | 0.15 | |
| Total Iloprost | 0 | 4.6 | 3.8 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 22B
Treprostinil Impurities Summary for 10 μg/mL
Iloprost, 600 μg/mL Treprostinil, pH 6.5, 115
mM NaCl at 40 ± 2° C./75 ± 5% RH, 3 mL LDPE ampules

Interval (Months)

| Relative Retention Time | T = 0 | 1 | 2 | 3 |
|---|---|---|---|---|
| T RRT~1.308 | ND | ND | 0.10 | |
| T RRT~1.308 | 0.18 | ND | ND | |
| T RRT~1.684 | 0.09 | ND | ND | |
| T RRT~1.757 | ND | ND | ND | |
| Total Treprostinil | 0.27 | 0.00 | 0.10 | |
| Unspecified impurities | | | | |

Report RRT and % adjusted area for all impurities ≥0.05% adjusted area
ND = Not Detected (<LOD)
NR = Not Reported (≥LOD and <0.05%)
NT = Not Tested

### Example 4

### Stability Testing of Iloprost/Treprostinil Formulation 2

TABLE 23
Stability Testing Summary for 20 μg/mL Iloprost, 600 μg/mL Treprostinil, pH
6.5, 115 mM NaCl at 25 ± 2° C./60 ± 5% RH, 5 mL glass vial Sample Orientation: Inverted

| | Testing Interval (Months) | | | | | | |
|---|---|---|---|---|---|---|---|
| Attribute (Test Method) | T = 0 | | 1 | | 2 | | 3 |
| Appearance | Clear, Colorless | | Clear, Colorless | | Clear, Colorless | | |
| | solution, essentially | | solution, essentially | | solution, essentially | | |
| | free from visible | | free from visible | | free from visible | | |
| | particulate matter. | | particulate matter. | | particulate matter. | | |
| pH USP <791> | 6.6 | | 6.6 | | 6.6 | | |
| Osmolality (mOsm/kg) | 315 | mOsm/kg | 318 | mOsm/kg | 316 | mOsm/kg | |
| USP <785> | | | | | | | |
| Iloprost Assay (% LC) | 97.1% | LC | 95.4% | LC | 94.1% | LC | |
| “Fit for Purpose” | | | | | | | |
| Treprostinil Assay (% LC) | 97.5% | LC | 96.9% | LC | 97.9% | LC | |
| “Fit for Purpose” | | | | | | | |

TABLE 24A
Iloprosi Impurities Summary for 20 μg/mL Iloprosi,
600 μg/mL Treprostinil, pH 6.5, 115 mM NaCl
at 25 ± 2° C./60 ± 5% RH, 5 mL glass vial

Interval (Months)

| Relative Retention Time | T = 0 | 1 | 2 | 3 |
|---|---|---|---|---|
| RRT~0.059 | ND | 0.93 | ND | |
| RRT~0.214-0.220 | 0.36 | 0.12 | 0.10 | |
| RRT~0.435 | ND | ND | 0.20 | |
| RRT~0.500 | ND | 0.06 | ND | |
| RRT~0.584 | ND | ND | 0.21 | |
| RRT~0.916 | ND | 0.06 | ND | |
| Total Iloprost | 0.36 | 1.2 | 0.51 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 24B
Treprostinil Impurities Summary for 20 μg/mL
Iloprost, 600 μg/mL Treprostinil, pH 6 5, 115
mM NaCl at 25 ± 2° C./60 ± 5% RH, 5 mL glass vial

Interval (Months)

| Relative Retention Time | T = 0 | 1 | 2 | 3 |
|---|---|---|---|---|
| T RRT~1.308 | ND | ND | 0.10 | |
| T RRT~1.308 | 0.18 | 0.11 | 0.08 | |
| T RRT~1.684 | 0.09 | 0.06 | 0.06 | |
| T RRT~1.757 | ND | ND | ND | |
| Total Treprostinil | 0.27 | 0.11 | 0.24 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 25
Stability Testing Summary for 20 μg/mL Iloprost, 600 μg/mL Treprostinil, pH
6.5, 1.15 mM NaCl at 40 ± 2° C./75 ± 5% RH, 5 mL glass vial Sample Orientation: Inverted

| | Testing Interval (Months) | | | | | | |
|---|---|---|---|---|---|---|---|
| Attribute (Test Method) | T = 0 | | 1 | | 2 | | 3 |
| Appearance | Clear, Colorless | | Clear, Colorless | | Clear, Colorless | | |
| | solution, essentially | | solution, essentially | | solution, essentially | | |
| | free from visible | | free from visible | | free from visible | | |
| | particulate matter. | | particulate matter. | | particulate matter. | | |
| pH USP <791> | 6.6 | | 6.6 | | 6.6 | | |
| Osmolality (mOsm/ke) | 315 | mOsm/kg | 319 | mOsm/kg | 317 | mOsm/kg | |
| USP <785> | | | | | | | |
| Iloprost Assay (% LC) | 97.1% | LC | 97.2% | LC | 93.7% | LC | |
| “Fit for Purpose” | | | | | | | |
| Treprostinil Assay (% LC) | 97.5% | LC | 96.9% | LC | 98.2% | LC | |
| “Fit for Purpose” | | | | | | | |

TABLE 26A
Iloprost Impurities Summary for 20 μg/mL Iloprost,
600 μg/mL Treprostinil, pH 6.5, 115 mM NaCl
at 40 ± 2° C./75 ± 5% RH, 5 mL glass vial

| | |
|---|---|
| Relative Retention | Interval (Months) |

| Time | T = 0 | 1 | 2 | 3 |
|---|---|---|---|---|
| RRT~0.059 | ND | 0.78 | ND | |
| RRT~0.158 | ND | ND | 0.08 | |
| RRT~0.214-0.220 | 0.36 | 0.19 | 0.10 | |
| RRT~0.418 | ND | 0.28 | ND | |
| RRT~0.435 | ND | ND | 0.35 | |
| RRT~0.462 | ND | 0.05 | ND | |
| RRT~0.584 | ND | ND | 0.38 | |
| RRT~0.743 | ND | ND | 0.22 | |
| Total Iloprost | 0.36 | 1.2 | 1.1 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 26B
Treprostinil Impurities Summary for 20 μg/mL Iloprost, 600
μg/mL Treprostinil, pH 6.5, 115 mM NaCl at 40 ± 2° C./75 ± 5% RH, 5
mL glass vial

Interval (Months)

| Relative Retention Time | T = 0 | 1 | 2 | 3 |
|---|---|---|---|---|
| T RRT~1.017 | ND | ND | 0.10 | |
| T RRT~1.308 | 0.18 | ND | ND | |
| T RRT~1.684 | 0 09 | 0.06 | 0.06 | |
| T RRT~1.757 | ND | ND | ND | |
| Total Treprostinil | 0.27 | 0.06 | 0.16 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 27
Stability Testing Summary for 20 μg/mL Iloprost, 600 μg/mL Treprostinil,
pH 6.5, 115 mM NaCl at 25 ± 2° C./60 ± 5% RH, 3 mL LDPE ampules

| | Testing Interval (Months) | | | | | | |
|---|---|---|---|---|---|---|---|
| Attribute (Test Method) | T = 0 | | 1 | | 2 | | 3 |
| Appearance | Clear, Colorless | | Clear, Colorless | | Clear, Colorless | | |
| | solution, essentially | | solution, essentially | | solution, essentially | | |
| | free from visible | | free from visible | | free from visible | | |
| | particulate matter. | | particulate matter. | | particulate matter. | | |
| pH USP <791> | 6.6 | | 6.6 | | 6.6 | | |
| Osmolality (mOsm/kg) | 315 | mOsm/kg | 315 | mOsm/kg | 314 | mOsm/kg | |
| USP <785> | | | | | | | |
| Iloprost Assay (% LC) | 97.1% | LC | 97.8% | LC | 94.2% | LC | |
| “Fit for Purpose” | | | | | | | |
| Treprostinil Assay (% LC) | 97.5% | LC | 96.9% | LC | 98.1% | LC | |
| “Fit for Purpose” | | | | | | | |

TABLE 28A
Iloprost Impurities Summary for 20 μg/mL Iloprost,
600 μg/mL Treprostinil, pH 6.5, 115 mM NaCl
at 25 ± 2° C./60 ± 5% RH, 3 mL LDPE ampules

Interval (Months)

| Relative Retention Time | T = 0 | 1 | 2 | 3 |
|---|---|---|---|---|
| RRT~0.059 | ND | 0.83 | ND | |
| RRT~0.078 | ND | 0.08 | ND | |
| RRT~0.128 | ND | 0.18 | ND | |
| RRT~0.214-0.220 | 0.36 | 0.13 | 0.11 | |
| RRT~0.397 | ND | 0.05 | ND | |
| RRT~0.418 | ND | 0.34 | ND | |
| RRT~0.435 | ND | ND | 0.24 | |
| RRT~0 454-0.457 | ND | 0.07 | 0.09 | |
| RRT~0.462 | ND | 0.24 | ND | |
| RRT~0.500 | ND | 0.28 | ND | |
| RRT~0.623 | ND | 0.14 | ND | |
| RRT~0.654 | ND | 0.2 | ND | |
| RRT~0.707 | ND | ND | 0.06 | |
| RRT~0.820 | ND | ND | 0.12 | |
| RRT~0.877 | ND | 0.07 | ND | |
| RRT~0.939 | ND | 0.22 | ND | |
| Total Iloprost | 0.36 | 2.8 | 0.62 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 28B
Treprostinil Impurities Summary for 20 μg/mL Iloprost, 600
μg/mL Treprostinil, pH 6.5, 115 mM NaCl at 25 ± 2° C./60 ± 5% RH, 3
mL LDPE ampules

Interval (Months)

| Relative Retention Time | T = 0 | 1 | 2 | 3 |
|---|---|---|---|---|
| T RRT~1.017 | ND | ND | 0.10 | |
| T RRT~1.308 | 0.18 | 0.07 | 0.06 | |
| T RRT~1.684 | 0.09 | ND | ND | |
| T RRT~1.757 | ND | ND | ND | |
| Total Treprostinil | 0.27 | 0.07 | 0.15 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 29
Stability Testing Summary for 20 μg/mL Iloprost, 600 μg/mL Treprostinil,
pH 6.5, 115 mM NaCl at 40 ± 2° C./75 ± 5% RH, 3 mL LDPE ampules

| | Testing Interval (Months) | | | | | | |
|---|---|---|---|---|---|---|---|
| Attribute (Test Method) | T = 0 | | I | | 2 | | 3 |
| Appearance | Clear, Colorless | | Clear, Colorless | | Clear, Colorless | | |
| | solution, essentially | | solution, essentially | | solution, essentially | | |
| | free from visible | | free from visible | | free from visible | | |
| | particulate matter. | | particulate matter. | | particulate matter. | | |
| pH USP <791> | 6.6 | | 6.6 | | 6.6 | | |
| Osmolality (mOsm/kg) | 315 | mOsm/kg | 314 | mOsm/kg | 310 | mOsm/kg | |
| USP 785 | | | | | | | |
| Iloprost Assay (% LC) | 97.1% | LC | 97.9% | LC | 94.4% | LC | |
| “Fit for Purpose” | | | | | | | |
| Treprostinil Assay (% LC) | 97.5% | LC | 97.6% | LC | 98.6% | LC | |
| “Fit for Purpose” | | | | | | | |

TABLE 30A
Iloprost Impurities Summary for 20 μg/mL Iloprost,
600 μg/mL Treprostinil, pH 6.5, 115 mM NaCl
at 40 ± 2° C./75 ± 5% RH, 3 mL LDPE ampules

| | |
|---|---|
| Relative Retention | Interval (Months) |

| Time | T = 0 | 1 | 2 | 3 |
|---|---|---|---|---|
| RRT~0.059-0.062 | ND | 0.73 | 0.59 | |
| RRT~0.078 | ND | 0.07 | ND | |
| RRT~0.122-0.127 | ND | 0.06 | 0.09 | |
| RRT~0.128 | ND | 0.13 | ND | |
| RRT~0.214-0.220 | 0.36 | 0.14 | 0.12 | |
| RRT~0.405 | ND | 0.07 | ND | |
| RRT~0.418 | ND | 0.41 | ND | |
| RRT~0.435 | ND | ND | 0.56 | |
| RRT~0.445 | ND | 0.26 | ND | |
| RRT~0.454- 0.457 | ND | ND | 0.25 | |
| RRT~0.462 | ND | 0.29 | ND | |
| RRT~0.471 | ND | 0.18 | ND | |
| RRT~0.500 | ND | 0.17 | ND | |
| RRT~0.623 | ND | 0.06 | ND | |
| RRT~0.653-0.654 | ND | 0.22 | 0.05 | |
| RRT~0.692 | ND | 0.09 | ND | |
| RRT~0.707 | ND | ND | 0.10 | |
| RRT~0.724 | ND | ND | 0.06 | |
| RRT~0.820 | ND | ND | 0.13 | |
| Total Iloprost | 0.36 | 2.9 | 1.9 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

TABLE 30B
Treprostinil Impurities Summary for 20 μg/mL
Iloprost, 600 μg/mL Treprostinil, pH 6.5. 115
mM NaCl at 40 ± 2° C./75 ± 5% RH, 3 mL LDPE ampules

Interval (Months)

| Relative Retention Time | T = 0 | 1 | 2 | 3 |
|---|---|---|---|---|
| T RRT~1.017 | ND | ND | 0.10 | |
| T RRT~1.308 | 0.18 | ND | ND | |
| T RRT~1.684 | 0 09 | ND | ND | |
| T RRT~1.757 | ND | ND | ND | |
| Total Treprostinil | 0.27 | 0.00 | 0.10 | |
| Unspecified Impurities | | | | |

Report RRT and % adjusted area for all impurities ≥0.05% adjusted area
ND = Not Detected (<LOD)
NR = Not Reported (≥LOD and <0.05%)
NT = Not Tested

### CONCLUSION

The stability testing results in Examples 2-4 indicate that iloprost is surprising more stable in the treprostinil-containing formulations 1 and 2 of Examples 3 and 4 compared to the formulation of Example 2, which did not contain treprostinil. Without being bound by any theory, these result may indicate that the iloprost molecule may be attaching to the Treprostinil molecule in the formulation 1 and 2 of Examples 3 and 4.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A method of treating pulmonary hypertension comprising administering by inhalation to a subject in need thereof iloprost or a pharmaceutically acceptable salt thereof and treprostinil or a pharmaceutically acceptable salt thereof.

2-21. (canceled)

22. A method of treating pulmonary hypertension comprising administering by inhalation to a subject in need thereof in a single event dose a composition comprising (i) iloprost or a pharmaceutically acceptable salt thereof and (ii) treprostinil or a pharmaceutically acceptable salt thereof, wherein the dosage of the iloprost or a pharmaceutically acceptable salt thereof delivered to the subject is at least 2.5 μg, and wherein the dosage of treprostinil or a pharmaceutically acceptable salt thereof delivered to the subject is at least 5 μg.

23-36. (canceled)

37. A pharmaceutical formulation comprising iloprost or a pharmaceutically acceptable salt thereof and treprostinil or a pharmaceutically acceptable salt thereof.

38. The pharmaceutical formulation of claim 37, which is a liquid formulation.

39. The pharmaceutical formulation of claim 36, which is an inhalable formulation.

40. The pharmaceutical formulation of claim 38, wherein a concentration of the iloprost in the formulation is from 5 μg/ml to 50 μg/ml.

41. (canceled)

42. The pharmaceutical formulation of claim 38, wherein a concentration of the treprostinil in the formulation is 200 μg/ml to 2000 μg/ml.

43. (canceled)

44. The pharmaceutical formulation of claim 38, wherein the formulation further comprises a buffer.

45. The pharmaceutical formulation of claim 44, wherein the buffer comprises sodium phosphate buffer.

46. (canceled)

47. The pharmaceutical formulation of claim 38, wherein the formulation further comprises a salt.

48. The pharmaceutical formulation of claim 47, wherein the salt is sodium chloride.

49-50. (canceled)

51. The pharmaceutical formulation of claim 38, wherein the formulation is an isotonic solution.

52. The pharmaceutical formulation of claim 38 having a pH from about 6.0 to 7.0.

53. (canceled)

54. The pharmaceutical formulation of claim 38, wherein the formulation has an osmolality from 250 mOsm/kg to 400 mOsm/kg.

55. (canceled)

56. The pharmaceutical formulation of claim 37, wherein the iloprost or a pharmaceutically acceptable salt thereof is iloprost or the tromethamine salt of iloprost.

57. The pharmaceutical formulation of claim 37, wherein the treprostinil or a pharmaceutically acceptable salt thereof is treprostinil or the sodium salt of treprostinil.

58. A method of treating pulmonary hypertension comprising administering by inhalation to a subject in need thereof the pharmaceutical formulation of claim 37.

59-74. (canceled)

75. The method of claim 58, further comprising preparing the pharmaceutical formulation; storing the prepared pharmaceutical formulation for a storage period of at least one month after the preparing, and wherein said administering is performed after said storing.

76-80. (canceled)

81. The method of claim 75, wherein an amount of the iloprost in the formulation after the storing is at least 90% of an amount of the iloprost in the formulation before the storing.

82. A dosage form comprising a dosage container and the pharmaceutical formulation of claim 37 in the container.

83-84. (canceled)
